(19) United States
(12) Patent Application Publication
(10) Pub. No.: US 2003/0045485 A1
(43) Pub. Date: Mar. 6, 2003

(54) P53-MEDIATED APOPTOSIS FOR THE THERAPEUTIC TREATMENT OF DISEASES

(76) Inventors: MICHAEL F. CLARKE, ANN ARBOR, MI (US); JAMES J. RYAN, SALINE, MI (US); GABRIEL NUNEZ, ANN ARBOR, MI (US)

Correspondence Address: Medlen & Carroll, LLP, 101 Howard Street, Suite 350, San Francisco, CA, 94105, US

(21) Appl. No.: 08/463,069

(22) Filed: Jun. 5, 1995

Related U.S. Application Data
(63) Continuation of application No. 08/139,301, filed on Oct. 19, 1993, now abandoned.

(51) Int. Cl. A61K 48/00; C12Q 1/68; C07H 21/04; C12P 21/02; C12N 5/06

(52) U.S. Cl. 514/44; 424/93.21; 424/93.1; 536/24.5; 435/344; 435/6; 435/69.1; 435/320.1

(57) ABSTRACT

The invention is directed to a methods of reducing the viability of a proliferating mammalian cells such as cancer cells. In one method cells deficient in p53 activity and in p53 suppressor activity of one or more p53-interacting regulatory proteins cell viability is reduced by increasing the level or activity of p53 in the cell. In another method viability of cells exhibiting p53 activity and p53 suppressor activity of one or more p53-interacting regulatory proteins is reduced by reducing the suppressor activity of the one or more p53-interacting regulatory proteins. Further, cell viability is reduced in cells deficient in p53 activity and exhibiting p53 suppressor activity of one or more p53-interacting regulatory proteins by a method that includes: (a) increasing the level or activity of p53 in the cell, and (b) reducing the suppressor activity of the one or more p53-interacting regulatory proteins. Also, included are methods of selectively reducing the viability of proliferating cancer cells compared to nonproliferating normal cells within a mixed population of cells and to methods of selectively reducing the viability of chronic granulocytic leukemia cells within a sample of proliferating bone marrow cells.

Throughout this application various publications are referenced within parentheses. The disclosures of these publications in their entireties are hereby incorporated by reference in this application in order to more fully describe the state of the art to which this invention pertains.

### BACKGROUND OF THE INVENTION

This invention relates generally to the regulation of cell growth and differentiation and, more particularly, to the modulation of cell cycle regulatory proteins for the therapeutic treatment of uncontrolled cell growth.

It is becoming increasingly apparent that carcinogenesis is the result of the deregulation of the intricate control mechanisms which have evolved in multicellular organisms to control normal cell growth and differentiation. The protein products of oncogenes, first identified as the acute transforming genes transduced by retroviruses, are a group of dominantly acting genes, including growth factors and their receptors, as well as second messengers and mitogenic nuclear proteins activated by growth factors, involved in cell division. The molecular cloning and characterization of the tumor suppressor genes p53 and Rb, or retinoblastoma, shows that both of these proteins suppress cell growth.

It has recently become apparent that normal hematopoiesis is dependent not only on pathways which positively or negatively regulate cell growth, but also on factors which regulate apoptosis, or programmed cell death. Growth factors are necessary for both lymphoid and myeloid progenitor cell survival, and certain cellular and viral transforming genes function to prevent apoptosis.

The standard methods of treatment for cancer currently include surgery, radiation therapy, and chemotherapy using cytotoxic drugs. However, for therapy to be effective, essentially all cancer cells need to be eradicated. Even if a very small fraction of the original population remains, over time, the cancer can reappear. An additional problem in treating cancer results from metastasis. Thus, removal of a primary tumor is only effective if done before any metastasis has occurred. High dose chemotherapy +/− radiation therapy followed by bone marrow transplantation is one approach to the treatment of metastatic cancer. However, certain cancers in which high dose chemotherapy might be useful, i.e., small cell cancer of the lung and breast cancer, commonly metastasize to the bone marrow. Thus, further techniques to rid the bone marrow samples of tumor cells are needed so tumor cells are not reinfused with the bone marrow.

In an effort to improve cancer therapy, treatments have now included the use of gene therapy to inhibit cellular growth. Expression of regulatory proteins such as p53 and Rb in animal models has been shown to inhibit proliferation of cancer cells and to modestly prolong the animals life. However, such treatments require long-term expression in essentially every abnormal cell. Current methods cannot achieve this requirement. Moreover, expression of these regulatory proteins has only been shown to inhibit proliferation, and not to effect cell viability as would be preferred for the most effective form of treatment.

Thus, there exists a long-felt need for a more efficient and less toxic means of cancer treatment which results in the loss of cell viability. The present invention satisfies these needs and provides related advantages as well.

### SUMMARY OF THE INVENTION

The invention is directed to a methods of reducing the viability of a proliferating mammalian cells such as cancer cells. In one method cells deficient in p53 activity and in p53 suppressor activity of one or more p53-interacting regulatory proteins cell viability is reduced by increasing the level or activity of p53 in the cell. In another method viability of cells exhibiting p53 activity and p53 suppressor activity of one or more p53-interacting regulatory proteins is reduced by reducing the suppressor activity of the one or more p53-interacting regulatory proteins. Further, cell viability is reduced in cells deficient in p53 activity and exhibiting p53 suppressor activity of one or more p53-interacting regulatory proteins by a method that includes: (a) increasing the level or activity of p53 in the cell, and (b) reducing the suppressor activity of the one or more p53-interacting regulatory proteins. Also, included are methods of selectively reducing the viability of proliferating cancer cells compared to non-proliferating normal cells within a mixed population of cells and to methods of selectively reducing the viability of chronic granulocytic leukemia cells within a sample of proliferating bone marrow cells.

### BRIEF DESCRIPTION OF THE DRAWINGS

FIG. 1 shows RNA and protein analysis of p53^{ts}neo transfectants.

FIG. 2 shows the loss of viability induced by wild-type p53.

FIG. 3 shows DNA fragmentation in p53 induced apoptosis.

FIG. 4 shows cell cycle analysis by quantitative flow cytometry of clone C9.

FIG. 5 shows loss of viability induced by wild-type p53.

FIG. 6 shows cell cycle analysis by quantitative flow cytometry.

FIG. 7 shows DNA fragmentation correlates with commitment to cell death.

FIG. 8 shows viability and differentiation of DP16-1 MEL cells grown in media containing 1.6% DMSO.

FIG. 9 shows DMSO induces translocation of p₅₃^{ts} into the nucleus and subsequent apoptosis of p53^{ts}neo transfected DP16-1 cells.

FIG. 10 shows analysis of cells for expression of the exogenous p53 and c-myb transcripts.

FIG. 11 shows viability and differentiation of c-myb transfected cells grown in media containing DMSO.

FIG. 12 shows the coimmunoprecipitation of c-myb and p53.

FIG. 13 shows Expression of c-myc in p53^{ts}neo transfected DP16-1 cells.

FIG. 14 shows the analysis of exogenous c-myc mRNA and p53 mRNA.

FIG. 15 shows conformation of p53^{ts} in DMSO-treated DP16-1 cells.

FIG. 16 shows the viability of DP16-1 cells transfected with c-myc with or without mutant p53

FIG. 17 shows the differentiation of DP16-1 cells transformed with p53 and c-myc during DMSO-induced differentiation.

### DETAILED DESCRIPTION OF THE INVENTION

This invention is directed to simple and effective methods for controlling and preventing unwanted cell growth. The methods exploit the genetic differences between proliferating cells such as cancer, for example, and normal cells and utilize the functions of cell cycle regulatory proteins to specifically kill only the proliferating cells. This genetic selectivity offers several advantages over conventual cancer therapies. For example, specific genetically based compounds can be used to modulate uncontrolled cell growth which are markedly less toxic than conventual treatments and thus, eliminate or reduce damage to normal tissues. Moreover, the methods are amenable to the selective killing of cancer cells which are often resistant to conventual treatment. Finally, the novel methods described herein offer the additional advantage of augmenting the efficacy of conventual therapy by combining the two approaches.

In one embodiment, proliferating tumor cells are specifically killed over non-proliferating normal cells by inducing apoptosis using the tumor suppressor gene product p53. In proliferating tumor cells that are deficient in both p53 and other cell cycle regulatory proteins that can modulate the apoptotic function of p53, specific killing is accomplished by directly expressing a functional p53 protein. An adenovirus-derived vector is used to deliver and express a functional p53 encoding nucleic acid by infection of cells in the proliferating area. Since apoptotic induction is rapid, p53 expression can be accomplished by either transient or stable expression. Viability of normal, non-proliferative cells expressing p53 will unchanged, but, proliferating tumor cells will be arrested in G1 of the cell cycle and under go apoptotic cell death.

In another embodiment, proliferating tumor cells that express functional p53 and also express the above mentioned p53-interacting cell cycle regulatory proteins are specifically killed by eliminating the function of the interacting proteins. Inhibition of the cell cycle regulatory proteins releases p53 from their suppressing activity and allows p53-induced apoptosis to occur in the proliferating cells. Function is eliminated using antisense oligonucleotides to one or more of the p53-interacting proteins. Alternatively, adenovirus-mediated gene transfer can be employed to express antisense nucleotide sequences within the proliferating cells, or to introduce a dominant-negative form of the p53-interacting protein.

In yet another embodiment, proliferating tumor cells deficient in p53 function but expressing p53-interacting regulatory proteins are specifically killed by first expressing p53 through adenovirus-mediated gene transfer and then selectively inhibiting expression of the p53-interacting cell cycle regulatory protein or proteins. Inhibition is again accomplished by administering antisense oligonucleotides or a dominant-negative form of the p53-interacting protein to decrease expression or activity of the p53-interacting proteins.

In still a further embodiment, p53-induced apoptosis is used to purge chronic granulocytic leukemia (CML) from bone marrow samples that are to be used for autologous bone marrow transplants. As with the selective killing of proliferating cells to control uncontrolled cell growth, manipulation of the expression of p53 and cell cycle regulatory proteins is used to selectively kill CML in low density mononuclear bon marrow cells isolated from a patient with CML. The bone marrow cells are first treated with macrophage inflammatory factor (MIP-1α) to arrest normal hematopoietic cells in G0. Arrested cells are not susceptible to p53-induced apoptosis whereas, in the functional absence of p53-interacting cell cycle regulatory proteins, proliferating cells are susceptible to p53-mediated cell death. Induction of p53-mediated apoptosis is accomplished by inhibiting the suppressor effects of such interacting cell cycle regulatory proteins by culturing the bone marrow cells in the presence of c-myb antisense oligonucleotides. CML cells, which are unresponsive to MIP-1α, will continue to cycle and are committed to apoptotic cell death.

As used herein, the term “p53” or “p53 tumor suppressor gene product” refers to a polypeptide having a molecular weight of 53,000 kD and is a nuclear regulatory protein. The p53 nucleotide and deduced amino acid sequences have been described for a broad range of species and are well known in the art. A description of such sequences can be found in, for example, Baker et al., Science 244:217-221 (1989); Bischoff et al., Mol. Cell Biol. 12:1405-1411 (1992) and Chin et al., Science 255:459-462 (1992), all of which are herein incorporated by reference. Nucleic acids encoding a p53 tumor suppressor gene product are available within the art or can be obtained by isolating a cDNA using the published sequences and standard methods within the art. It is understood that limited modifications may be made without destroying the biological activity of p53, and that only a portion of the entire primary structure may be required in order to effect activity. These modifications may be deliberate, as through site-directed mutagenesis, or may be accidental such as through mutation in hosts which are p53 producers. All of these modifications are included as long as p53 activity is retained. Further, various molecules can be attached to p53, for example, other proteins, carbohydrates, or lipids. Such modifications are included within the definition of p53.

As used herein, the term “p53 activity” refers to the biological functions of the p53 tumor suppressor gene product. It is understood that proteins exhibiting similar biological function but having different nucleotide and amino acid sequences than p53 or a p53 homolog, are also intended to fall within the definition of proteins, or cells expressing proteins having p53 activity. The biological functions of p53 include, for example, the suppression of cell growth, such as that described by Friend et al., Nature 323: 643-646 (1986), and the induction of programmed cell death or apoptosis. Induction of cell death through p53 is specific to proliferating cells and can be suppressed by cell cycle regulatory proteins. Thus, the term “p53-mediated apoptosis” as used herein refers to the commitment of proliferating cells to programmed cell death that is caused by the presence of p53 activity.

As used herein, the term “p53 suppressor activity” refers to activity mediated by, for example, a protein that is capable of reducing or inhibiting one or more biological functions of p53. Examples of proteins exhibiting p53 suppressor activity include cell cycle regulatory proteins that can bind p53 and prevent apoptosis. Such proteins can include, for example, c-myb, c-myc and bc12. Thus, the use of the term “p53-interacting regulatory proteins” is intended to refer to those cell cycle regulatory proteins, for example, that reduce or inhibit the activity of p53 and p53-like proteins. Modification of p53 activity can be by direct interaction, such as by the physical binding of p53, or can be indirect, such as by the functional modulation of p53 activity through a p53 regulatory pathway.

As used herein, the term “expressing” refers to the biosynthesis of a nucleic acid or polypeptide through endogenous cellular mechanisms. The term includes all necessary steps for the transcription and/or the transcription and translation of a genetic sequence into the nucleic acid or polypeptide. The genetic sequence can be a natural sequence or be derived by recombinant or by synthetic means. Also included are any regulation, processing and sorting events required to achieve a desired outcome, such as splicing of introns and post translational modifications.

As used herein, the term “substantially” when used in reference to nucleotide sequence complimentarity refers to the degree to which two sequences are complimentary so as to allow specific hybridization under a given set of conditions. Hybridization principles and methods for determining hybridization specificity are well established and are known by one skilled in the art. Such principles and methods can be found in, for example, Sambrook et al., Molecular Cloning: A Laboratory Manual, Cold Spring Harbor Laboratory, New York (1992), and in Ausubel et al., Current Protocols in Molecular Biology, John Wiley and Sons, Baltimore, Md. (1989), both of which are herein incorporated by reference. Thus, it is not necessary that two nucleic acids exhibit sequence identity to be substantially complimentary, only that they can specifically hybridize or be made to specifically hybridize without detectible cross reactivity with other similar sequences.

As used herein, the term “effective amount” refers to the amount of an agent or compound required to obtain a desired outcome. For example, if a compound is required to arrest proliferation, than the amount added to the cells that produces this response is considered to be an effective amount. One skilled in the art knows, or can determine what an effective amount is for a particular cell type or tissue, or, what constitutes an effective amount for a therapeutic treatment.

As used herein, the term “biologic response modifier” refers to a peptide, polypeptide or protein that is capable of altering cellular growth and differentiation functions. Such molecules can be of natural or of synthetic origin so long as they are capable of changing a particular growth or differentiation function in a predetermined way. Examples of biologic response modifiers include growth factors and mitogens which are known to either inhibit or promote these functions. One specific biologic response modifier which is capable of arresting cell proliferation is macrophage inflammatory factor (MIP-1α), for example. Macromolecules and compounds other than peptides, polypeptides and proteins are also intended to be included within the definition. Such other macromolecules and compounds can include, for example, carbohydrates, lipids, amino acids and drugs such as mimosine.

Cellular growth and differentiation is controlled by numerous distinct as well as interacting processes. The difference between normal and uncontrolled cell growth and differentiation relies on the accurate regulation of these processes. Numerous proteins and their encoding genes have been identified that play a role in one or more growth and differentiation processes. For example, tumor suppressor gene products, such as p53 and Rb, suppress cell proliferation, Friend et al., supra and Marshall, C. J. Cell 64:313-326 (1991). Deficiencies in these gene products can lead to uncontrolled cell growth, such as cancer, for example. A specific example is in the case of erythroleukemia and erythroleukemia cell lines where there is generally a lack of expression, or the expression of a mutant p53 tumor suppressor gene product.

The involvement of p53 in cellular control can be complex and depend on the cell type and state of differentiation. As stated above, myeloid leukemic cell lines frequently fail to express p53, however, lymphoid leukemic cell lines characteristically overexpress this protein. Moreover, some lines that express normal, or wild type, p53 such as ML 1, for example, lack expression during logarithmic growth but show increased levels with induced differentiation. Further, mouse erythroleukemia cell lines (MEL) expressing mutant p53 typically show a decrease in expression with induced differentiation. p53 also plays a role in the control of proliferation of early myeloid precursors.

The results generated in the examples which follow demonstrate that expression of p53 protein in erythroleukemia cells is associated with growth arrest and that this arrest occurs in actively cycling populations of cells predominantly in the G0-G1 transition and G1 phase of the cell cycle. In addition, cell death occurs rapidly following p53 induced growth arrest in proliferating cells but not in non-cycling or differentiated cells. The cell death induced by p53 is accompanied by endonucleolytic cleavage of genomic DNA characteristic of apoptosis. Importantly, p53 induced cell death is not solely a function of its ability to block cells in G1 indicating that p53 expression affects cell viability independent of growth arrest directly attributable to p53. Thus, p53 has two related but separable roles as a cell cycle regulatory protein; the control of cell proliferation and the control of cell viability.

In addition to p53, other cell cycle regulatory proteins exist that can control growth and differentiation processes. These cell cycle regulatory proteins include, for example, the nuclear proteins c-myb and c-myc and non-nuclear proteins such as bc12. Also, members of the tyrosine kinase family of oncogenes can be included as cell cycle regulatory proteins. This family includes, for example, src, ras, grb2, raf and bcr/abl. For the specific case of hematopoietic cells, expression of c-myb and c-myc are essential for entry into S-phase of the cell cycle and for DNA synthesis, (Yonish-Roach et al., Nature 352:345 (1991). In addition to their role in cell cycle transit in normal cells, constitutive expression of c-mb also blocks MEL cell differentiation whereas expression of c-myc in arrested cells induces apoptosis. Numerous other gene products known within the art also exhibit cell cycle regulatory functions. One example is the bc12 gene product which functions to promote cell viability by blocking apoptosis.

As described here and below, there are several cell cycle regulatory proteins that are able to modify the p53-mediated apoptosis of proliferating cells. These p53-interacting regulatory proteins, which include those described above, either alleviate or delay p53-induced cell death. For example, c-myb and c-myc both partially inhibit p53-mediated apoptosis whereas bc12 delays this process. Additionally, mdm2 binds and inactivates p53 to a significant extent.

Conversely, just as p53-interacting regulatory proteins modify p53-mediated apoptosis, p53 can also modify the function of these regularly proteins. For example, the c-myb dependent differentiation block in MEL cells is overcome in the presence of p53 just as the c-myc dependent apoptosis is inhibited in the presence of p53. Moreover, many tumor cells contain a mutant form of p53 that is unable to induce apoptosis, however, still retains its ability to disrupt myc-induced apoptosis of arrested cells. Thus, the interaction of cell cycle regulatory proteins, whether tumor suppressor genes or oncogenes, is complex with each protein modifying the phenotypic effects of the other in unexpected ways.

The invention provides a method of reducing the viability of a proliferating mammalian cell deficient in p53 activity and in p53 suppressor activity of one or more p53-interacting regulatory proteins. The method includes increasing the level or activity of p53 in the proliferating mammalian cell.

The invention also provides a method of reducing the viability of a proliferating mammalian cell exhibiting p53 activity and p53 suppressor activity of one or more p53-interacting regulatory proteins. The method includes reducing the suppressor activity of the one or more p53-interacting regulatory proteins.

The invention further provides a method of reducing the viability of a proliferating mammalian cell deficient in p53 activity and exhibiting p53 suppressor activity of one or more p53-interacting regulatory proteins. The method includes increasing the level or activity of p53 in the cell, and reducing the suppressor activity of the one or more p53-interacting regulatory proteins.

The above described functions of cell cycle regulatory proteins can be advantageously used to regulate uncontrolled cell growth of proliferating mammalian cells by inducing p53-mediated apoptosis in the proliferating cell or population. Proliferating mammalian cells susceptible to p53-mediated apoptosis include, for example, cancer cells and other neoplasia, particularly of the hematopoietic cell lineage. Cancer cells other than of the hematopoietic lineage can include, for example, cell types as diverse as lymphoma cells, breast cancer cells, prostate cancer cells, oat cell carcinoma cells, lung cancer cells, colon cancer cells, bladder cancer cells, brain tumor cells, head and neck cancer cells, and pancreatic cancer cells. Numerous other cell types susceptible to p53-mediated apoptosis exist as well and are known, or can be determined by one skilled in the art. Thus the invention provides for a method of reducing the viability of a proliferating mammalian cell wherein the cell is any one of a variety of cancer cell types.

The method by which cell viability can be reduced will depend on the genetic background of the cell as to whether they exhibit p53 activity and/or p53 suppressor activity and whether the cell is actively proliferating. In the case where cells are deficient in both of these activities, cell death can be induced by increasing the level or activity of p53. Such increases can be obtained by introducing into the cell an expression vector containing a p53 encoding nucleic acid. Expression of the p53 gene product in a proliferating cell will lead to growth arrest in the G1 phase of the cell cycle and result in apoptotic cell death. Non-proliferating or arrested cells, such as normal terminally differentiated cells, for example, will be unaffected by the expression of p53.

In the opposite case where the cells exhibit both p53 activity and p53 suppressor activity, cell viability can be reduced by inhibiting or decreasing the suppressor activity. Inhibition of p53 suppressor activity essentially produces cells exhibiting only p53 activity and the cells are therefore susceptible to p53-mediated apoptosis. Again, p53 activity will only lead to cell death in proliferating cells and not in arrested cells.

The inhibition of p53 suppressor activity can be obtained by a variety of methods. Such methods include, for example, directly inhibiting suppressor activity by treating the cells with compounds or agents that bind to and inactivate molecules, such as p53-interacting regulatory proteins, that exhibit the p53 suppressor activity. Another methodology includes introduction into the cell of a dominant-negative mutant of the p53-interacting regulatory proteins. Such mutants inhibit the function of their wild-type counterparts. A specific example of a dominant-negative mutant of a p53-interacting regulatory protein is mbm2.

Alternatively, indirect inhibition of suppressor activity can be obtained by using agents such as growth factors, growth factor agonists or antagonists, antibodies and the like that modulate the levels of the p53-interacting proteins. Another method is to inhibit the synthesis of such proteins through the use of antisense or triplex oligonucleotides, analogues or expression constructs. This method entails introducing into the cell a nucleic acid sufficiently complementary in sequence so as to specifically hybridize to the target p53-interacting regulatory protein encoding gene or message. Triplex inhibition relies on the transcriptional inhibition of the target gene and can be extremely efficient since only a few copies per cell are required to achieve complete inhibition. Antisense methodology on the other hand inhibits the normal processing, translation or half-life of the target message. Such methods are well known to one skilled in the art.

In the transitional case where cells are deficient in p53 activity but exhibit p53 suppressor activity, cell viability can be reduced by a combination of increasing the level of p53 activity and also reducing the level of p53 suppressor activity. Methods employed to achieve these conditions are identical to those described above. Again, the end result is the generation of cells that exhibit essentially only p53 activity.

Recombinant methods known in the art can be used to achieve any of the desired outcomes described previously for the generation of a cell susceptible to p53-mediated apoptosis. For example, vectors containing p53 encoding or c-myb, c-myc or bc12 antisense nucleic acids can be employed to express protein or antisense message and thereby elevate the level or activity of p53 or reduce the p53 suppressor activity. Such vectors are known or can be constructed by those skilled in the art and should contain all expression elements necessary to achieve the transcription of antisense, or transcription, translation, regulation, and sorting of the protein. Other beneficial characteristics can also be contained within the vectors such as mechanisms for recovery of the nucleic acids in a different form. Phagemids are a specific example of this because they can be used either as plasmids or as bacteriophage vectors. Examples of other vectors include viruses, such as bacteriophages, baculoviruses and retroviruses, cosmids, plasmids, liposomes and other recombination vectors. The vectors can also contain elements for use in either procaryotic or eucaryotic host systems. One of ordinary skill in the art will know which host systems are compatible with a particular vector.

The vectors can be introduced into cultured cells by any one of a variety of known methods within the art. Such methods can be found described in Sambrook et al., Molecular Cloning: A Laboratory Manual, Cold Spring Harbor Laboratory, New York (1992), in Ausubel et al., Current Protocols in Molecular Biology, John Wiley and Sons, Baltimore, Md. (1989), and include, for example, stable or transient transfection, lipofection, electroporation and infection with recombinant viral vectors. Introduction of nucleic acids by infection offers several advantages over the other listed methods which includes their use in both in vitro and in vivo settings. Higher efficiency can also be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of a viral vector for introducing p53 encoding or other appropriate nucleic acids for the induction of p53-mediated apoptosis is the adenovirus derived vector Adenop53TK. This vector expresses a herpes virus thymidine kinase (TK) gene for either positive or negative selection and the p53 gene. Alternatively, nucleic acids other, or in addition to the p53 gene, can be placed in the vector to effect p53-mediated apoptosis. Such other nucleic acids include antisense sequences to p53-interacting regulatory proteins. This vector can be used to infect cells that have an adenovirus receptor which includes most cancers of epithelial origin.

The above described vector as well as others that exhibit similar desired functions can be used to treat a mixed population of cells to selectively promote death of uncontrolled proliferating cells compared to normal cells. A mixed population of cells can include, for example, an in vitro or ex vivo culture of cells, a tissue or a human subject. Thus, the invention provides a method of selectively reducing the viability of proliferating cancer cells compared to non-proliferating normal cells within a mixed population of cells. The method includes selectively inducing p53-mediated apoptosis in said proliferating cancer cells.

As with the methods described previously, the ability of p53 activity to specifically kill growing, but not resting cells can be used for the therapeutic treatment of cancer. In order to successfully treat a cancer using gene therapy by inducing p53-mediated apoptosis, it is necessary to deliver the gene product to most or all of the target cancer cells. Also required is the essentially complete inhibition of p53 suppressor activity where appropriate by, for example, antisense oligonucleotides or expression. Thus, the genetic background of the cancer cells will determine whether exogenous p53 activity and/or inhibition of p53 suppressor activity is required. Since p53 induction of cell death is rapid, requiring as little as a 4-6 hour exposure to p53 during the G1 phase of the cell cycle, the expression of p53 need only be transient. Therefore, vectors that are efficient at infecting and transiently expressing a protein, such as the adenovirus vector above is advantageous for successful therapy.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce mutant forms of p53, cellular transformation will not occur. Moreover, features that limit expression to particular cell types can also be included. Such features include, for example, promoter and expression elements that are specific for the desired cell type.

A variety of modes can be used to administer the vectors for the specific induction of p53-mediated apoptosis within a diverse number of cancer cell types. Types of caners include, for example, localized tumors as well as diffuse soft tissue types. The mode of administration will depend on the location and type of cancer to be treated. For example, localized breast cancers or head and neck cancers can be treated by inoculation into they vascular system supplying the tumor with nutrients. This mode as well as subcutaneous injection can also be used to treat other types of cancers wherein the procedure takes advantage of vector target specificity. Alternatively, local administration by, for example, direct inoculation at the site of tumor growth, can provide a quicker and more effective treatment. Local administration, or inoculation into an artery that directly supplies the area of tumor growth, is advantageous because there is minimal dilution effect and therefore a smaller dose is required to achieve expression in a majority of the targeted cells. Other modes of administration can be used as well. Such modes are known and one skilled in the art will know which type to use to treat a particular type of cancer.

Specific examples of treating cancer through p53-mediated apoptosis using the above described gene therapy methods include, for example, subjects with breast or colon cancer metastasis to the liver. Such subjects can have the vector delivered to tumors through an hepatic artery infusion via a pump and indwelling catheter. Bladder tumors can be treated via infusion of vector into the bladder during cystoscopy and regional lymph nodes can be treated through injection into regional lymphatic vessels, for example. Other types of tumor can be treated as well. Moreover, an additional advantage of the described gene therapy methods is that they can be used in conjunction with traditional cancer therapies to further enhance eradication of the disease.

The invention provides a method of selectively reducing the viability of chronic granulocytic leukemia cells within a sample of proliferating bone marrow cells. The method includes (a) selectively arresting the proliferation of normal cells within the sample of proliferating bone marrow cells to generate a sample of bone marrow cells containing arrested normal cells and proliferating chronic granulocytic leukemia cells, and (b) inducing p53-mediated apoptosis in the proliferating chronic granulocytic leukemia cells.

Chronic myeloid leukemia (CML) is a slowly progressive disease that eventually results in death. One form of treating this disease is to purge bone marrow samples (autologous bone marrow) ex vivo of tumor cells before reinfusion into a subject in an autologous bone marrow transplant. The techniques currently employed for bone marrow purging, unfortunately, are time consuming and not entirely efficient. Furthermore, they result in some loss of stem cells. Clinical methods for preparing bone marrow or peripheral blood hematopoietic cells for autologous bone marrow transplantation are known in the art. (Deisseroth, A. B. et al., Human Gene Therapy, 2:359 (1991)).

Depending on whether the chronic granulocytic leukemia cells (CML) exhibit p53 activity and/or p53 suppressor activity, any of the above described methods can be use to effectively kill CML cancer cells within the population of bone marrow cells. One requirement to selectively reduce the viability of only CML cells however, is that the proliferation of the normal cells be arrested. Hematopoietic stem cells within the population are quiescent in G0 are not susceptible to p53-mediated apoptosis.

Biologic response modifiers, such as growth factors and cytokines are one means to achieve this specificity. A specific example of a biologic response modifier that can be use to specifically arrest normal hematopoietic cells is macrophage inflammatory factor (MIP-1α). This factor specifically arrests normal cells in the G0 phase of the cell cycle. Thus, a population of bone marrow cells can be treated with an amount of MIP-1α that arrests normal cells and then p53-mediated apoptosis can be induced in the susceptible proliferating CML cells. Effective amounts of biologic response modifiers are known within the art such as those described for MIP-1α (Graham et al., Nature 344: 442-444 (1990); Lord et al., Blood 79: 2605-2609 (1992)), or can be determined by one skilled in the art by titrating the modifier and determining at which concentration growth of normal cells is inhibited.

p53-mediated apoptosis can be induced using the viral vector described previously, for example, to express either a p53 encoding nucleic acid or antisense to one or more p53-interacting regulatory proteins or both. Alternatively, CML cells exhibiting, or made to exhibit, p53 activity can be treated with antisense oligonucleotides or analogues. Oligonucleotide analogues provide an additional advantage because they can be made to penetrate cellular membranes without the need for specific transfection procedures. This advantage applies to either in vivo or ex vivo treatment, or to in vitro cultures. A specific example of an membrane permeable oligonucleotide analog is phosphothioate oligodeoxynucleotides.

It is understood that modifications which do not substantially affect the activity of the various embodiments of this invention are also included within the definition of the invention provided herein.

The following examples are intended to illustrate but not limit the present invention.

### EXAMPLE I

### Plasmid Construction

p53^{ts}neo was constructed from plasmid pLTRp53cG (Eliyahu et al., Nature (London) 316:158 (1985)). A Bam HI site found in p53 intron sequences of pLTRp53cG was destroyed by partial digestion with Bam HI, and blunt ending with the large fragment of E. coli DNA polymerase I in a buffer containing all four deoxynucleotides. The plasmid was then recircularized with T4 DNA ligase. A 2.7 kb Nde I/Bam HI fragment from pSV-2neo containing the neomycin resistance gene, was inserted into the remaining Bam HI site of pLTRp53cG using Ban HI linkers. The 5′ Bam HI site was subsequently destroyed; p53^{ts}neo contains the coding sequence of the neomycin resistance gene inserted in the same orientation as the p53 coding sequences.

### EXAMPLE II

### p53-Mediated Apoptosis

Given the lack of expression of wild-type p53 in many MEL cell lines, to study the effect of wild-type p53 expression on MEL cell differentiation, an expression plasmid containing a minigene encoding a temperature sensitive p53, p53^{ts}neo (Ryan et al., supra), was electroporated into a p53-negative MEL cell line DP16-1, a MEL cell line which has no endogenous expression of p53 protein (Munroe et al., Mol. Cell. Biol. 10:3307 (1990); Mowat et al., Nature 314:633 (1985)). This mutant p53 contains a single base-pair mutation (alanine−−−>valine at position 135) and can cooperate with ras in transforming primary rat embryo fibroblasts at 37.5° C. At 37.5° C. the inactive ts-mutant p53^{ts} is predominantly cytoplasmic and is associated with the cellular heat shock protein hsc70. Clones expressing the mutant p53 grow rapidly at 37.5° C., however transfer to 32.5° C. leads to growth arrest and subsequent loss of viability. In cells grown at 32.5° C., p53^{ts} becomes located in the nucleus (Ginsburg et al., Mol. Cell. Biol. 11:582 (1991)) and this mutant behaves as wild-type p53 as shown by its inhibition of growth and oncogene-mediated transformation (Michalovitz et al., Cell 62:671 (1990); Michalovitz et al., Mol. Cell. Biol. 11:582 (1991)).

DP16-1 MEL cells were grown in Dulbecco modified minimal essential medium (DMEM) supplemented with 10% calf serum, 2 mM glutamine, 100 U of penicillin per ml, and 100 μg of amphotericin B per ml. DNA transfections were performed by electroporation (Prochownik et al., supra). Transfection mixes consisted of 1×10⁷ cells in 400 μl transfection buffer (272 mM sucrose, 7 mM phosphate (pH 7.4), 1 mM MgCl₂) with either 10 μg p53^{ts}neo, or 10 μg pSV-2neo, linearized with Bam HI. Transfected cells were selected in media containing 1 mg/ml geneticin (G418; GIBCO). Single-cell clones were isolated by limiting cell dilution in 96-well microtiter plates.

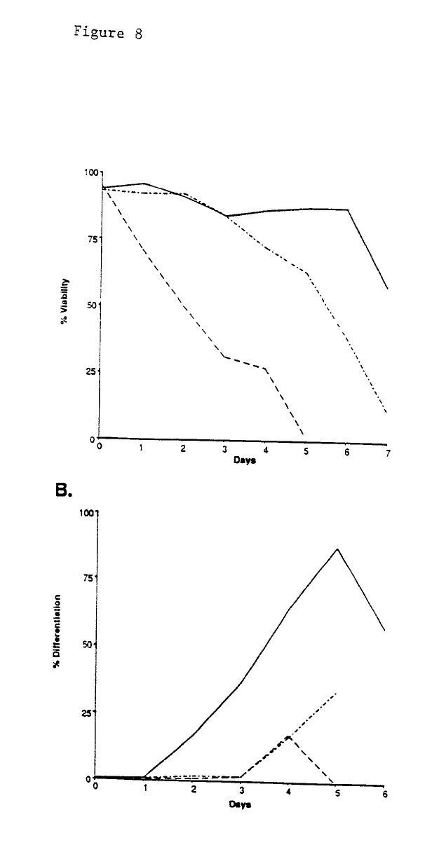

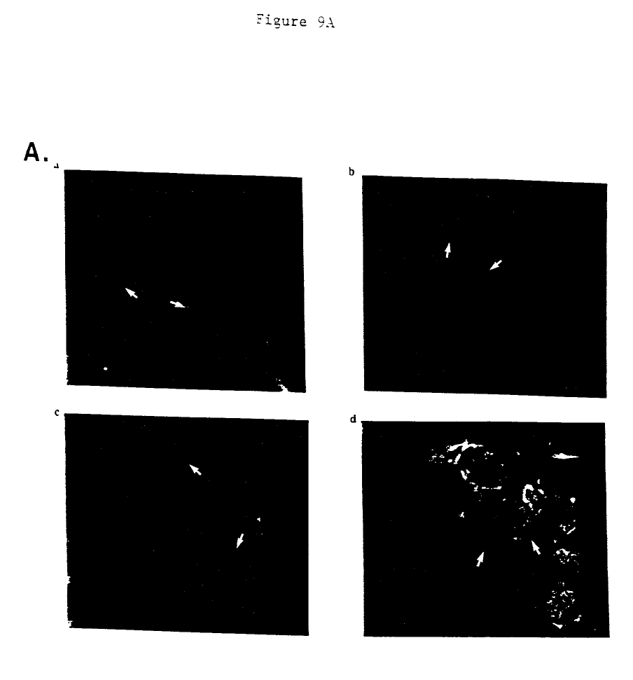

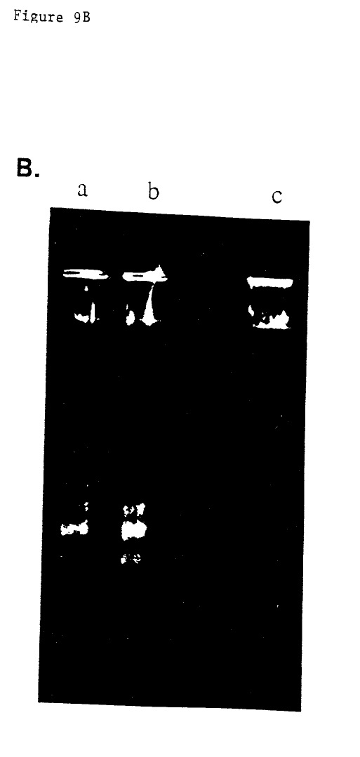

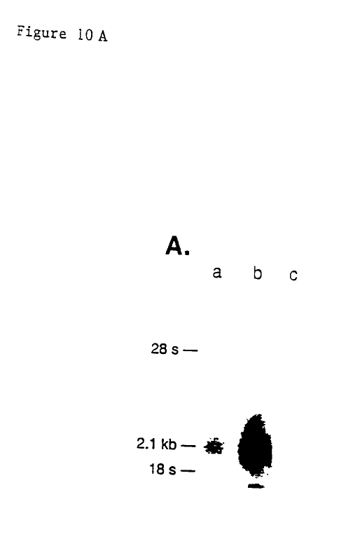

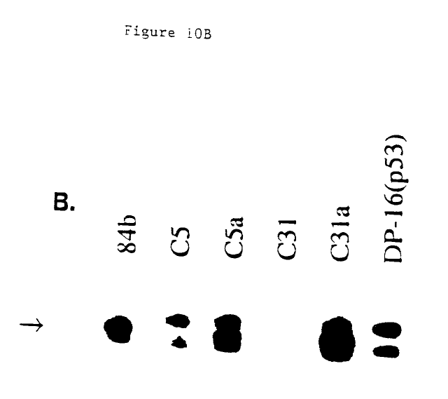

To characterize the consequences of enforced expression of p53 on differentiation, logarithmically growing transfectants were exposed to media containing 1.6% dimethyl sulfoxide (DMSO) and incubated at 37.5° C. Media was changed every other day. Cells underwent growth arrest and subsequently died over a period of 48-96 hours (FIG. 8A; DP16-1 cells, ______, p53^{ts}neo transfected DP16-1 cells, _{— — —}, and DP16-1 cells co-transfected with c-myb and p53^{ts}neo, _._._). Viable cells were isolated by centrifugation through Ficoll-paque (Pharmacia). Furthermore, benzidine staining for heme expression (Gusella et al., Cell 9:221 (1976)) revealed that only a small percentage of the p53^{ts}neo transfected cells had differentiated prior to cell death (FIG. 8B; legend as described for FIG. 8A). This is in contrast to untransfected control DP16-1 cells which, when grown in media containing DMSO, downregulate c-myb mRNA and differentiate while remaining viable (FIG. 8A & 8B). As expected, the p₅₃^{ts} protein was located in the cytoplasm of the cells growing at 37.5° C. in the absence of DMSO (FIG. 9A, panel a; C4 p53^{ts}neo DP16-1 cells). Surprisingly, even when grown at 37.5° C., exposure of the p53^{ts}neo transfected MEL cells to 1.6% DMSO led to the relocation of the p53^{ts} protein in the nucleus (FIG. 9A, panel b; C4 cells) followed by cell death, showing that DMSO can induce a mutant p53 to regain its function as a tumor suppressor. Genomic DNA isolated from such cells showed the oligonucleosomal “ladder” characteristic of apoptosis (FIG. 9B, lanes a & b; C4 and C9 p53^{ts}neo transfectants, respectively). In addition, analysis with conformation specific anti-p53 antibodies (Yewdell et al., J. Virology 59:444 (1986)) showed the majority of the p53^{ts} to be in the wild-type conformation. Thus despite the fact that the cells were grown at 37.5° C., DMSO caused the p₅₃^{ts} to behave as wild-type p53 tumor suppressor and induced apoptotic cell death.

### EXAMPLE III

### Nucleic Acid and Protein Analysis

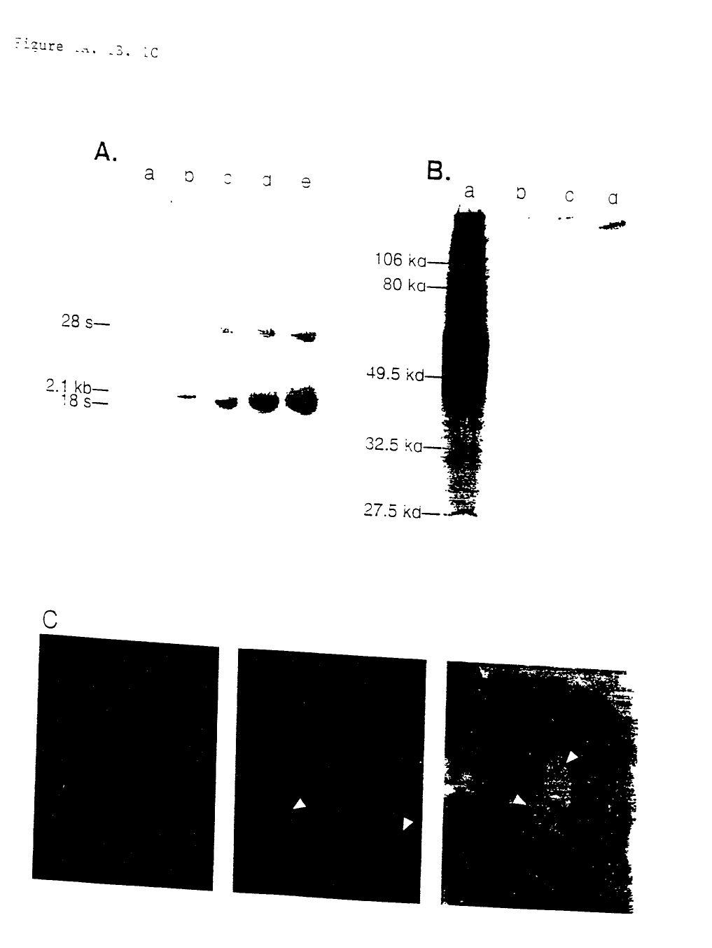

Total cellular RNA was isolated from clones growing at 37.5° C. and purified by the guanidinium thiocyanate/phenol extraction method (Chomczynski et al., Anal. Biochem. 162:156 (1987)). Northern blotting and hybridization were performed as previously described (Clarke et al., supra). To detect exogenous p53 expression the 3.5 kb Sma I fragment of p53^{ts}neo was used as probe, labelled with [α-³²P] dCTP (Amersham) by random priming using T7 DNA polymerase (Stratagene). FIG. 1A shows expression of p53 related message in the unselected p53^{ts}neo transfected bulk culture (lane e), as well as in three individual clones, C2, C4, and C9 (lanes b,c and d, respectively), isolated from the bulk culture. A transcript of approximately 2.1 kilobases is seen in the transfected clones. This transcript is not seen in the RNA from the parental cell line DP16-1 (lane a).

Genomic DNA was prepared as follows. Briefly, cells were suspended in a buffer consisting of 20 mM Tris, pH 7.5, 10 mM EDTA, 300 mM NaCl, lysed by adding SDS to 0.5%, digested with proteinase K, followed by organic extraction, and ethanol precipitation. DNA samples (3 μg) were analyzed by electrophoresis through a 1.5% agarose gel containing 0.5 μg/ml ethidium bromide, or through a non-denaturing 5% polyacrylamide gel.

To confirm the expression of p53 protein in transfected clones, cellular proteins were labelled by incubating cells (1×10⁷) for 3 hours with 0.2 mCi of [³⁵S]methionine in 1 ml of methionine-free DMEM. Cells were lysed in lysis buffer (150 mM NaCl, 1% triton X-100, 50 mM Tris (pH 8.0), 0.5 mM phenylmethylsulfonyl fluoride) and pre-cleared by overnight incubation at 4° C. with 50 μl of normal goat serum, followed by addition of protein G sepharose beads (Pharmacia) and centrifugation. The supernatant was immunoprecipitated with a mixture of PAb421 (Harlow et al., J. Virology 39:861 (1981)), a pan-specific monoclonal antibody against p53, and PAb248, a murine-specific monoclonal antibody against p53. Immune complexes were collected with protein A agarose beads (BRL), washed 3× in lysis buffer and suspended in 25 μl of Laemmli sample buffer. Samples were electrophoresed through 10% polyacrylamide in the presence of SDS and dried gels utilized for autoradiography. The p53 protein is clearly seen in the transfected clone C9, but not in parental line DP16-1 or in immunoprecipitations with control monoclonal antibody (FIG. 1B; clone C9 immunoprecipitated with p53 antibody (lane a), parental line DP16-1 immunoprecipitated with p53 antibody (lane b) and control monoclonal antibody (lane c), clone C9 immunoprecipitated with control monoclonal antibody (lane d). Molecular weights (kd) are shown.).

To prepare cells for immunohistochemical staining approximately 2.5×10⁵ cells were suspended in buffer (phosphate buffered saline (PBS) —Ca⁺⁺, −Mg⁺⁺, 1% bovine serum albumin fraction V, 0.5% tween-20, 5 μM EDTA) and pelleted on a microscope slide using a cytospin centrifuge. Cells were fixed with methanol for 60 minutes at 4° C. and then washed twice with PBS. 40 μl of a mixture of PAb421 and PAb248 was added to each slide and allowed to stand for 60 minutes at room temperature. Following a PBS wash, 25 μl of goat anti-mouse-FITC (Tago) was added for 60 minutes at room temperature. After a final PBS wash the cells were counterstained with Evans blue (Sigma) and viewed through fluorescence microscopy. Immunohistochemistry shows that the p53 protein is located in the cytoplasm (arrowheads) of the transfected cells, but not the parental cells, grown at 37.5° C., and is translocated to the nucleus when cells are incubated at 32.5° C. (FIG. 1C; DP16-1 cells (left) and DP16-1 cells transfected with p53^{ts}neo grown at 37.5° C. (middle) and 32.5° C. (right)).

### EXAMPLE IV

### Cell Cycle Analysis Of p53 Induced Cell Death

The level of p53 protein varies in a cell-cycle dependent manner in MEL cells (Khochbin et al., Exp. Cell Res. 179:565 (1988)). Low levels are seen early in G1, with a subsequent increase at the G1/S transition and a more moderate increase occurring during the remainder of S phase. A constant level is seen during G2/M, with a return to low levels following division.

The relationship of p53 induced cell death to the cell cycle was studied in transfected clone C9. Clone C9 cells were synchronized in G0/G1 by density arrest (typically at a concentration of 1-2×10⁶ cells/ml) for 48-72 hours at 37.5° C. Cells were stimulated to begin cycling by replating into fresh media at a 1:10 split ratio. Growth arrest with mimosine (Sigma) was accomplished by the addition of 300 μM mimosine to logarithmically growing cells for a period of 24 hours.

Isoleucine deficient media was prepared using a DMEM select-Amine Kit (Gibco). Logarithmically growing cells were arrested by transfer to isoleucine deficient media for a period of 18-24 hours.

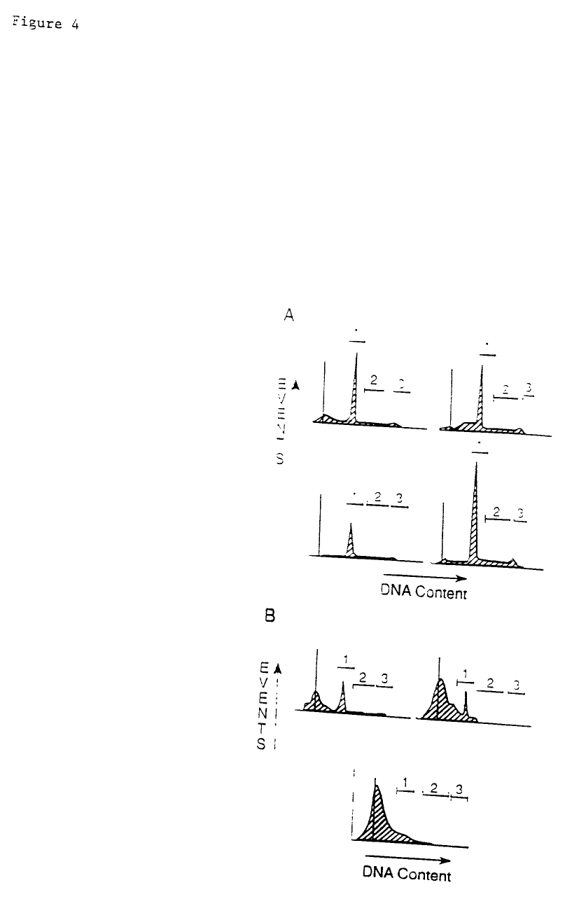

Following release from density arrest by splitting 1:10 in fresh media, the cells were placed at 37.5° C. and 32.5° C. At 37.5° C. the cells progressed into S and G2/M after a delay of approximately 20 hours (FIG. 4A). Briefly, cell cycle analysis performed at the following time-points after release from density arrest showed: 0 hours (top left) 85% G1, 11% S, and 4% G2/M; 6 hours (top right) 75% G1, 23% S, and 2% G2/M; 20 hours (bottom left) 55% G1, 44% S, and 1% G2/M; and 26 hours (bottom right) 50% G1, 44% S, and 6% G2/M. Cells released from density arrest at 32.5° C. fail to begin cycling secondary to a very rapid decrease in viability (FIG. 4B). The figure shows the cell cycle analysis done 6 hours (top left), 9 hours (top right), and 12 hours (bottom) after release from density arrest. Indicated are the DNA contents of avian erythrocytes (line) and MEL cells in the G1 (1), S (2), and G2/M (3) phases of the cell cycle. By 12 hours after release, the majority of cells remain in G0/G1 though the sharpness of the G0/G1 peak is lost as cell viability decreases.

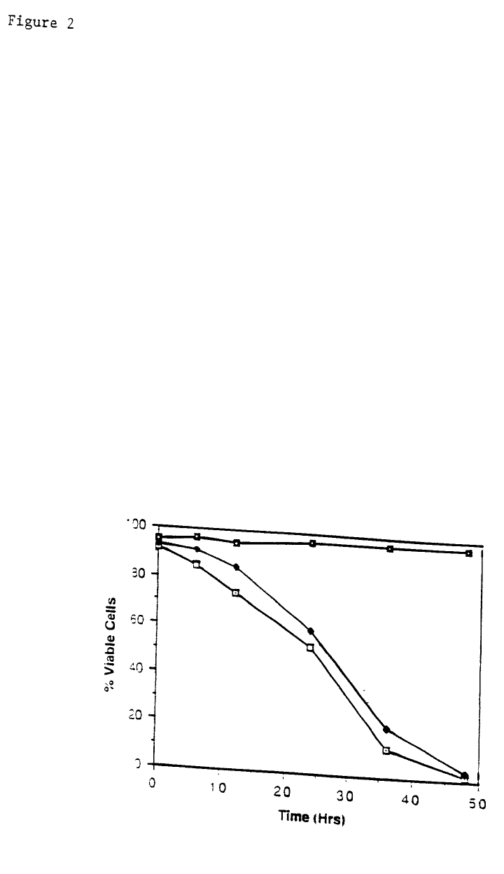

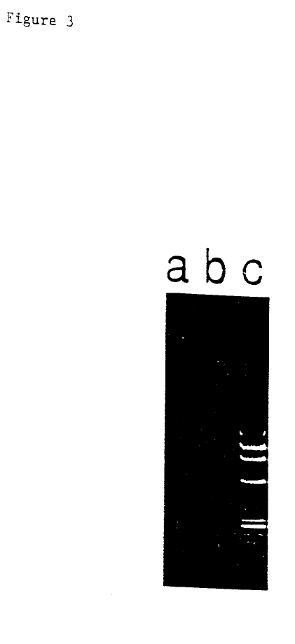

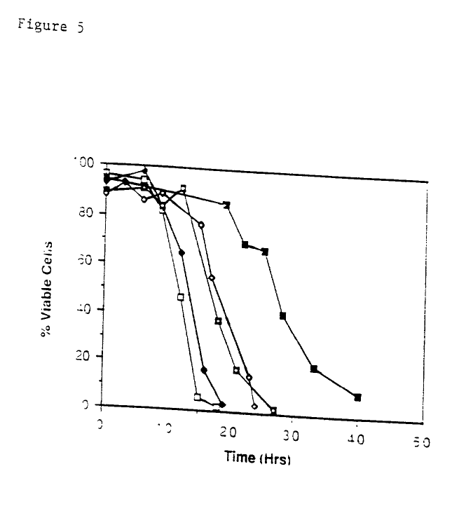

Comparison of the kinetics of cell death with that of a randomly growing population of clone C9 cells placed at 32.5° C. indicates that cells released into Gi following density arrest undergo cell death at an accelerated rate (FIG. 5; time at 37.5° C. was: [symbol] hours, ♦ 6 hours, [symbol] 9 hours, [symbol] 15 hours, ▪ 28 hours). Almost complete cell death occurs by 15 hours at 32.5° C., as opposed to 36-48 hours for a randomly growing population. This indicated that cells released into Gi may be preferentially susceptible to wild-type p53 induced cell death. All six p53^{ts}neo clones examined including, clones C2, C4, and C9, as well as the uncloned G418 resistant bulk culture, showed similar kinetics following transfer to 32.5° C. (FIG. 2; parental line [symbol] DP16-1. ♦ Clone C4. [symbol] Clone C9). Six pSV-2neo transfected clones, as well as the parental cell line DP16-1, showed no loss of viability when grown at 32.5° C.

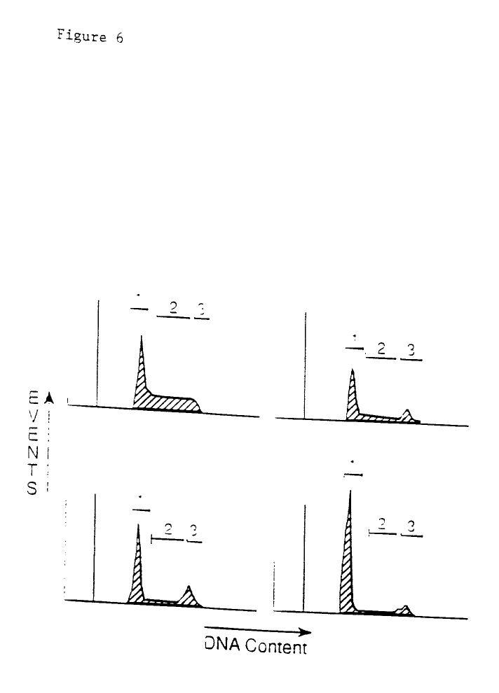

To explore the possibility that cells released into G1 may be preferentially susceptible to wild-type p53 induced death, media was added to density arrested cells and the cells were incubated for various times at 37.5° C., to allow progression out of GO. They were then placed at 32.5° C. and viability monitored. Cells released from density arrest at 37.5° C. for at least 12-15 hours prior to being placed at 32.5° C. began to show a survival advantage over cells released directly at 32.5° C. This advantage was more pronounced for cells allowed to progress for 24-48 hours at 37.5° C. (FIG. 5). Analysis of DNA content by quantitative flow cytometry of C9 cells released from density arrest at 37.5° C. for 36 hours prior to placement at 32.5° C. is shown in FIG. 6. Briefly, the cell cycle analysis of cells grown at 32.5° C. for the following times was: 0 hours (top left) 36% G1, 61.5% S, and 2.5% G2/M; 6 hours (top right) 47% G1, 33% S, and 20% G2/M; 15 hours (bottom left) 66.5% G1, 9% S, 24.5% G2/M; and 20 hours (bottom right) 81% G1, 8% S, and 11% G2/M. The DNA content of avian erythrocytes (vertical line) and MEL cells in the G1 (1), S (2), and G2/M (3) phases of the cell cycle are also shown.

What these results indicate is that at the time the cells were downshifted to 32.5° C. (0 hours), the majority of cells have progressed into S phase. However, after 6 hours at 32.5° C. the percentage of S phase cells has fallen significantly. This is attributable to a progression of some cells into G2/M, while at the same time the percentage of cells found in G1 also rises, indicating a failure of cells to progress from G1 to S. By 15 hours a majority of cells have accumulated in Gi. It is at this point that cell viability begins to decrease (FIG. 5). Cycling cells, therefore, progress through S phase and G2/M and accumulate in G1 prior to cell death occurring.

To rule out that G1 arrest alone induces cell death, clones C9, C2 and C1 were growth arrested with either mimosine (300 μM) (Watson et al., Cytometry 12:242 (1991)) or isoleucine deprivation (Heintz et al., Proc. Natl. Acad. Sci. USA 79:4083 (1982)) at 37.5° C., both of which result in late Gi arrest. These Gi arrested cells do not undergo rapid cell death, though viability decreases to approximately 70% after 3-4 days of arrest. Viability falls to less than 5% within 36 hours in cells arrested in Gi with either mimosine treatment or isoleucine-deprivation and subsequent culture at 32.5° C., indicating that cell death is specifically induced by wild-type p53 expression.

Cell cycle analysis was performed by fixing cells (1-2×10⁶ cells in PBS) with methanol, collecting by centrifugation, and resuspending the cells in propidium iodide (Sigma) stain (propidium iodide 50 μg/ml, 0.05% triton X-100, EDTA 18 μg/ml, RNase A 100 units/ml, in phosphate buffered saline (PBS)). Avian erythrocytes (1×10⁶) were utilized as an internal standard. After a 30 minute incubation at room temperature DNA content was determined by quantitative flow cytometry (Morasca et al., In (1986)).

### EXAMPLE V

### G0 and Differentiated Cells are not Subject to p53-Induced Cell Death

To determine if release into Gi was a necessary event for p53 to induce apoptosis, non-cycling GO cells were placed at 32.5° C. while still density arrested. Simultaneously, density arrested cells were kept at 37.5° C. and cells released into G1 (split 1:10) were placed at both 37.5° C. and 32.5° C. Viability was then monitored. While cells released into G1 at 37.5° C. grew well and maintained a high viability, cells released at 32.5° C. had the expected rapid loss of viability. At 37.5° C., density arrested p53^{ts}neo cells will lose viability slowly over time, and cell death is not accelerated at 32.5° C. The same cells split 1:10 undergo a much more rapid decline.

MEL cells can also exit the cell cycle by being stimulated to terminally differentiate. DMSO was used to induce differentiation of p53^{ts}neo clones at 37.5° C. After 5 days exposure to 1.6% DMSO, viable cells were isolated by centrifugation through Ficoll-paque. The cells obtained were terminally differentiated as shown by 1) high benzidine positivity (70-80%) and 2) failure to proliferate when placed at 37.5° C. in DMSO-free media. These cells were placed at 32.5° C. and viability monitored. Such cells maintained viability over several days, while control randomly growing cultures underwent the expected cell death.

### EXAMPLE VI

### Commitment to Cell Death is Cell Cycle Dependent

To delineate the time course of commitment to cell death, density arrested C9 cells were split 1:10 in fresh media and placed for varying periods of time at 32.5° C. They were then returned to incubation at 37.5° C. Viability was checked 18 hours after the cells had been released from density arrest. Cells which spent less than 3 hours at 32.5° C. maintained a high viability (Table 1). Between 3 and 4 hours, however, the majority of cells became committed to cell death, as shown by a low viability at 18 hours. By 6 hours at 32.5° C. approximately 90% of the cells are committed to undergoing cell death. Cells which remained viable at 18 hours were able to proliferate if kept at 37.5° C., indicating that they had escaped commitment to cell death.

### Table I

### Time Course of Commitment to p53 Induced Cell Death

Density arrested clone C9 cells were split 1:10 and placed at 32.5° C. for varying periods of time before transfer to 37.5° C. Viability was then determined 18 hours after release from density arrest.

| Time at 32.5° C. (hours) | Viability at 18 hours |
|---|---|
| 1 | 83% |
| 2 | 79% |
| 3 | 67% |
| 4 | 30% |
| 6 | 12% |

Density arrested clone C9 cells were split 1:10, allowed to cycle for 36 hours at 37.5° C., then placed at 32.5° C. for varying periods of time, prior to transfer to 37.5° C. Viability was determined 18 hours after placement at 32.5° C.

| Time at 32.5° C. (hours) | Viability at 18 hours |
|---|---|
| 4 | >90% |
| 6 | >90% |
| 8 | >90% |
| 10 | >90% |
| 18 | 68% |

In hematopoietic cells, p53 mRNA expression peaks in S phase (Khochbin et al., supra). Clone C9 cells which, following release from density arrest, were allowed to cycle at 37.50C for 36 hours into S phase (FIG. 6) do not show an early commitment to cell death when placed at 32.5° C. (Table 1). Although some cells have lost viability after 18 hours at 32.5° C., if returned to 37.5° C. many of the surviving cells will continue to proliferate. In contrast to cells in G1, where a brief exposure to p53 commits cells to die, even a prolonged exposure to wild-type p53 during S phase and G2/M does not appear to lead to an irreversible commitment to cell death.

### EXAMPLE VII

### DNA Fragmentation Occurs with Commitment to Cell Death

In order to understand the molecular events which lead to p53 induced MEL cell death, cells were analyzed for the appearance of DNA fragmentation. Cells were released from density arrest at 32.5° C., and genomic DNA was isolated at various time points.

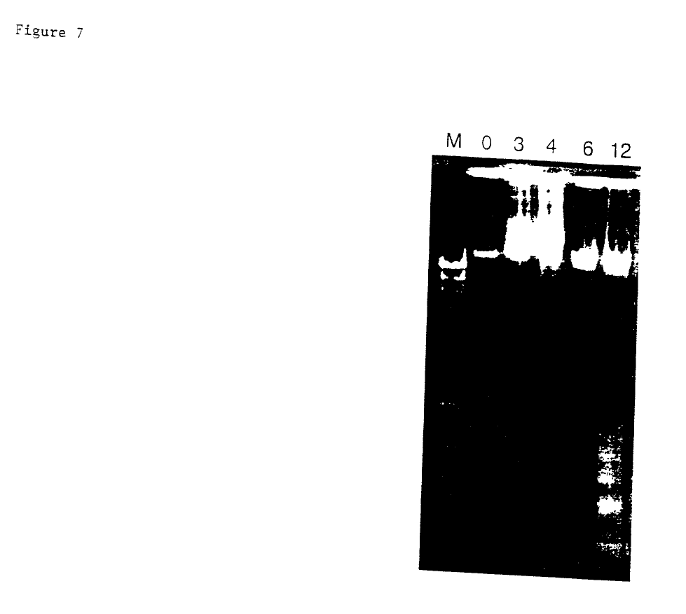

Commitment to cell death correlates with the initial appearance of low molecular weight DNA fragments (FIG. 7). The lanes are labelled in FIG. 7 with the number of hours cells were cultured at 32.5° C. prior to DNA isolation. Also shown is Hind III-digested lambda phage DNA and Hae III-digested PhiX174 phage DNA used as a molecular weight marker (M). This occurs after 4.5-6 hours at 32.5° C. (though cell viability remains greater than 90% at this time) in density arrested cells released into G1. Although appearing to be normal when viewed by light microscopy, the majority of these cells will lose viability despite being returned to 37.5° C.

### EXAMPLE VIII

### Integration of p53 and c-myb in DP16-1 Cells

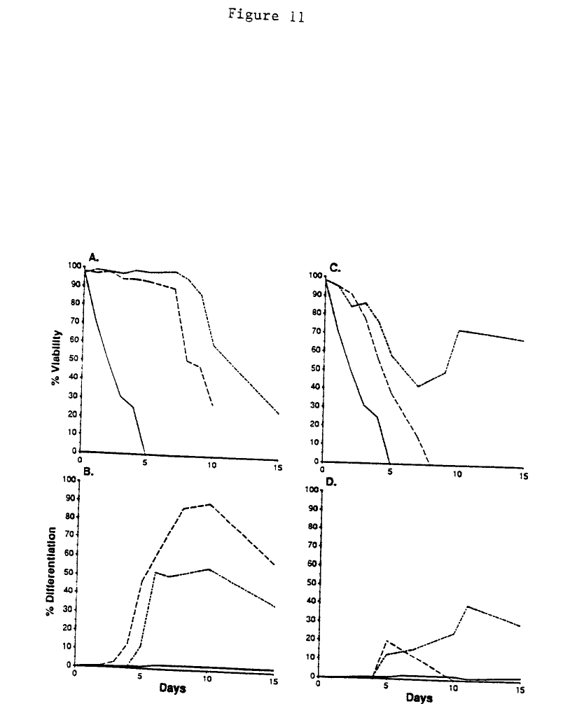

To determine how c-myb and p53 interact during MEL cell growth and differentiation, DP16-1 cells were co-transfected with p53^{ts}neo and pMbm1-dhfr, which encodes a full-length c-myb cDNA under the control of an SV40 promoter (Clarke, et al., supra). Whereas DP16-1 cells co-transfected with a control dhfr plasmid and p53^{ts}neo underwent apoptosis (FIG. 9B), MEL cells co-transfected with pMbml-dhfr and p53^{ts}neo did not undergo rapid cell death when exposed to 1.6% DMSO at 37.5° C. (FIG. 9A; panel c, C5a p53^{ts}neo/c-myb transfected DP16-1 cells; panel d, C5a cells). Instead, these cells proliferated for 2-3 days, and then arrested coincident with an increase in benzidine-positive cells (FIG. 11). Viability decreased only after the majority of cells had differentiated.

Increased expression of c-myb was selected for by growing transfected cells in higher concentrations of methotrexate (Clarke et al., supra). Whereas DP16-1 MEL cells transfected with pMbml-dhfr alone were highly resistant to DMSO-induced differentiation, amplified cultures of p53^{ts}neo/pMbm1-dhfr co-transfectants showed a significant degree of differentiation. These cultures also did not proliferate indefinitely, as do the pMbm1-dhfr transfectants, but instead lost viability after 10-14 days in 1.6% DMSO.

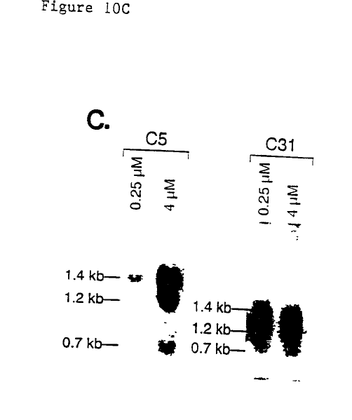

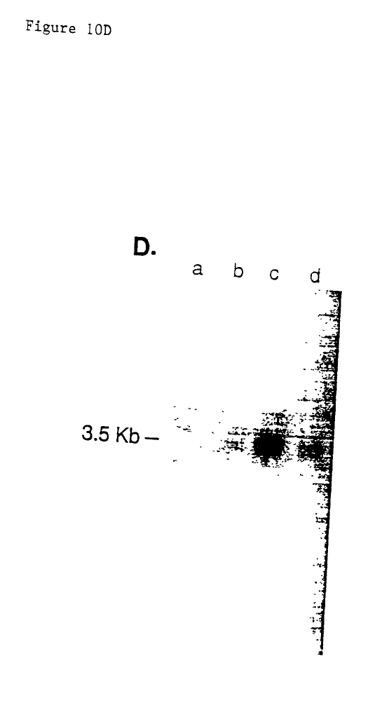

To further characterize the interactions of these genes, DP16-1 clones C5a and C31a, which expressed p53 (FIG. 10A; C5a (lane a), C31a (lane b), or DP16-1 (lane c)) and had significantly amplified c-myb mRNA levels, were isolated by limiting dilution. FIG. 10B shows c-myb RNA levels as determined by RNase mapping. C5 and C31 are clones grown in 0.25 μM methotrexate, and C5a and C31a are the respective clones grown in 4 μM methotrexate. The arrow shows the exogenous c-myb band. FIG. 10C shows the amplification of c-myb genomic DNA sequences (C5 (0.25 μM), C5a (4 μM), C31 (0.25 μM), and C31a (4 μM) cells) whereas FIG. 10D shows c-myb RNA levels as determined by northern blot (DP16-1 cells (lane a), 84B cells (lane b), C5a cells (lane c), and C31a cells (lane d)). To detect exogenous p53 expression of mRNA from clones C5a and C31a, the 3.5 kb Sma I fragment of p53^{ts}neo was used as probe, labeled with [α-³²P] dCTP (Amersham) by random priming using T7 DNA polymerase (Stratagene).

RNase mapping of c-myb was accomplished using total RNA (10 μg) incubated with 1×10⁵ cpm of ³²P labeled riboprobe for quantification of exogenous c-myb mRNA (Zinn et al., Cell 34:865 (1983)). The riboprobe vector was made by sub-cloning the Bam HI-Eco RI fragment of pMbm1 (Weber et al., supra) into pGEM 1. The resultant plasmid was digested with Pvu II, and a riboprobe generated using SP6 polymerase. The riboprobe and indicated mRNA was incubated at 50° C. for 16 hours, digested with RNase A and T1, and analyzed on a denaturing acrylamide gel (Zinn et al., supra). The undigested riboprobe is approximately 340 bases, and the Mbm1 specific message is 140 bases.

Amplification of the transfected c-myb DNAs was analyzed using genomic DNA that was isolated from C5 and C31 grown in 0.25 μM methotrexate, and C5a and C31a grown in 4 μM methotrexate was isolated by lysing cells in SDS, followed by proteinase K digestion, organic extraction and ethanol precipitation.

A 35 base oligodeoxynucleotide complimentary to bases 3271-3305 of human c-myb was labeled with T4 kinase and gamma ³²P ATP and used to probe a Northern blot to detect expression of human c-myb.

In contrast to cells transfected with p53^{ts}neo and dhfr, which rapidly die, C5 and C31 displayed intermediate levels of viability when exposed to media with DMSO (FIGS. 11A; p53^{ts}neo transfected DP16-1 cells, ______, C5 cells, _{— — —}, and C5a cells, _._._, & 11C; p53^{ts}neo transfected DP16-1 cells, ______, C31 cells, _{— — —}, and C31a cells, _. _. _). After further amplification of c-myb, the clones designated C5a and C31a, continued to grow when cultured in 1.6% DMSO at 37.5° C. (FIGS. 11A & 11C). Although there was some loss of viability, these clones continued to proliferate indefinitely despite the DMSO-induced translocation of p53 into the nucleus (FIG. 9, panel c & d). These data show that constitutive expression of high levels of c-mvb mRNA overcomes the anti-proliferative effect of p53.

### EXAMPLE IX

### p53 Inhibition of c-myb

Since c-myb is able to partially protect the cells from p53-induced apoptosis, it was next determined whether p53 has any effects on the c-myb differentiation block. In contrast to a control pMbm1/dhfr-transfected DP16-1 MEL cell culture, which exhibited the expected high level expression of c-mvb and resultant inhibition of differentiation, DP16-1 cells co-transfected with c-myb and p53 partially differentiated when grown in 1.6% DMSO. This implies that the presence of intranuclear p53 influences cells to differentiate despite the constitutive expression of c-myb. The extent of MEL cell differentiation is inversely related to the level of c-myb mRNA (Clarke et al., supra). Before amplification of c-myb, a high percentage of Cs and a moderate percentage of C31 cells could differentiate in response to DMSO (FIG. 11B; 84B cells, ______, C5 cells, _{— — —}, and C5a cells, _._._, and 11D; 84B cells, ______, C31 cells, _{— — —}, and C31a cells, _._._). In C31 cells, this lower level of differentiation was probably secondary to a more modest protection from apoptosis (FIG. 11C). After c-myb amplification, C5a and C31a cells still showed substantial levels of induced differentiation (FIGS. 11B & 11D), although the time course was delayed in comparison to the control DP16-1 cells (FIG. 8B).

In contrast, c-myb transfected DP16-1 cells and 84B cells, a MEL cell line constitutively expressing a similar level of c-myb mRNA as C5a and C31a (FIG. 11D), remained benzidine negative despite prolonged culture in 1.6% DMSO. As clone C5a and C31a cells were passaged in DMSO-containing media a significant percentage remained benzidine positive for several weeks. This indicates that new cells continued to become committed to terminal differentiation while the population as a whole retained a proliferative capacity. To confirm that these results were not unique to the DP16-1 MEL cell line, 84B MEL cells were transfected with p53. A clone was isolated, shown to express p53 and the exogenous c-myb, and induced to differentiate with DMSO. These cells behaved in a manner identical to C5a and C31a cells, i.e., a significant number of cells differentiated despite constitutive expression of c-myb. Thus, c-myb appears to delay differentiation in these cells, but, presumably because of p53 expression, does not block it completely.

### EXAMPLE X

Coimmunoprecipitation of p53 and c-myb

To determine whether the effects of co-expression of c-myb and p53 might be due to a direct interaction between the two proteins, it was determined whether antibodies to c-myb would coimmunoprecipitate p53.

C5a cells were seeded at a density of 4×10⁵ cells/ml and incubated at 32.5° C. for fourteen hours. Proteins were labeled as follows: 3×10⁶ cells were washed three times in serum and methionine free media, and incubated for three hours in 7 ml of methionine free DMEM, 10% dialyzed calf serum, and 150 μCi of 35S methionine. The cells were collected by centrifugation and lysed in 400 μl of RIPA buffer (10 mM Tris, pH 7.6, 1 mM EDTA, 150 mM NaCl, and 0.5 mM PMSF) with 0.2% triton X-100. The extracts were clarified by centrifugation at 12/,000×g for 10 minutes and used for co-immunoprecipitations.

Briefly, 5 μl of the first antiserum was added to 100 μl of sample, and samples were rocked at 4° C. for 4 hours followed by the additions of 30 μl of Staph G sepharose (Pharmacia) and incubated for an additional hour. The beads were pelleted in a microfuge for 10 seconds and washed 5 times in 800 μl of RIPA buffer with triton X-100. Protein complexes were disrupted by adding 400 μl of RIPA buffer with 0.1% SDS and 0.5% deoxycholate. The beads were pelleted, and the supernatants were transferred to new eppendorf tubes, centrifuged, and the supernatant again transferred to a new eppendorf tube. Twenty μl of anti-p53 monoclonal antibody (pAb 421) was then added, and the samples incubated overnight. Protein G sepharose beads were added, and samples incubated for an additional hour. The beads were then collected by centrifugation, and washed three times with RIPA buffer. After collecting the beads, they were resuspended in 25 μl of SDS sample buffer, and the bound proteins analyzed by SDS-PAGE and autoradiography.

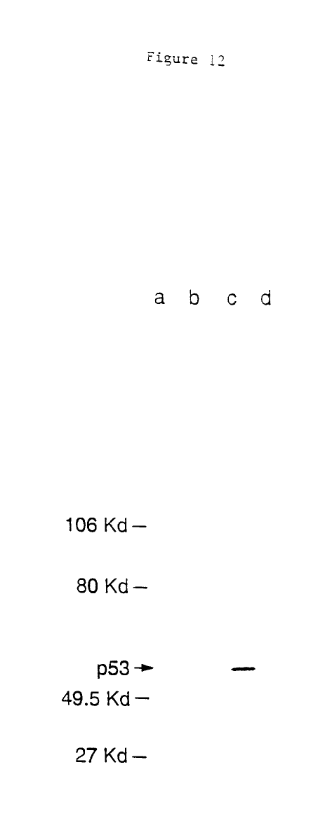

Shown in FIG. 12 are the following immunoprecipitations: lane a, 1st antibody was the pre-immune serum from the rabbit used to raise the anti-c-myb antibodies. Lane b, 1st antibody was serum from the rabbits immunized with recombinant bacterial anti-c-myb. Lane c, 1^{st} antibody was sheep anti-c-myb. Lane d, 1^{st} antibody was nonspecific goat serum. These results show that both c-myb and anti-sera, but not preimmune serum or non-specific serum, coimmunoprecipitated p53. Furthermore, the p53 protein was not coimmunoprecipitated with c-myb in 84B cells which do not express p53, or if a nonspecific antibody was used in place of the anti-p53 antibody for the second immunoprecipitation.

### EXAMPLE XI

### p53 Downregulates c-myc mRNA Levels

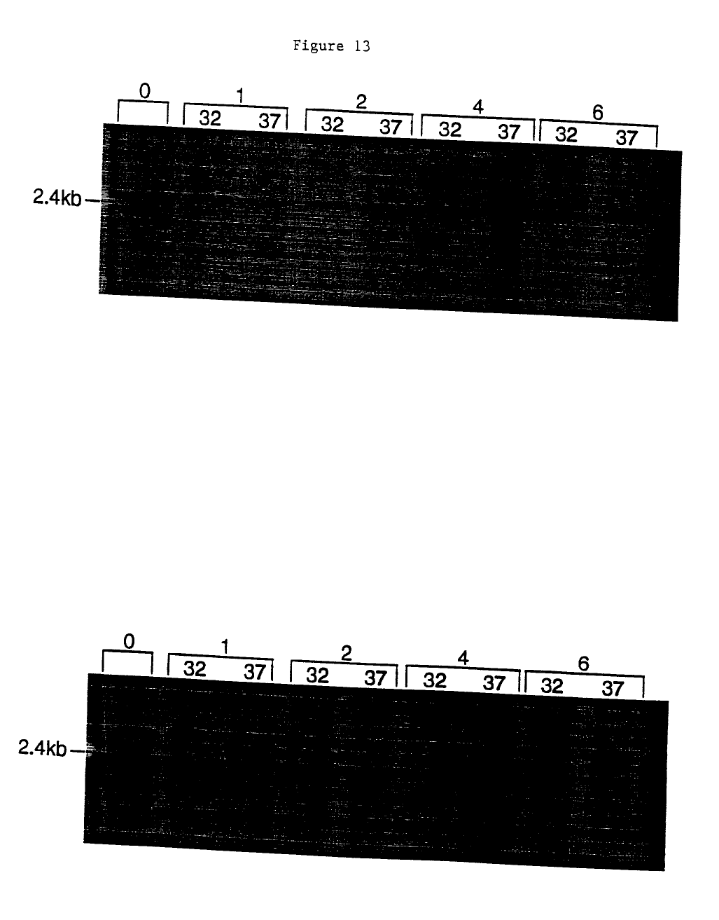

To determine the effects p53 has upon c-myc mRNA expression, cultures of p53^{ts}neo-transfected DP16-1 cells which had been density arrested for two days were released from density arrest either at 32.5° C. or 37.5° C. RNA was extracted at times zero, two, four, and six hours after release from density arrest. In two separate experiments, expression of the wild-type, but not mutant, p53 resulted in the downregulation of c-myc mRNA (FIG. 13).

DP16-1 cells were density arrested for two days. Cells, typically at a density of 1-2×10⁶ cells/ml, were then released from density arrest by diluting cells to a concentration of 4×10⁵ cells/ml with fresh medium. The cells were incubated at the indicated temperature for the indicated period of time. Cells were then harvested, and RNA was isolated by the method of Guanidine isothiocyanate lysis and CsTFA centrifugation. Twenty micrograms of RNA was electrophoresed through an agarose/formaldehyde gel and blotted onto a nylon membrane. Northern blots were probed with a mouse c-mvc cDNA Xho I fragment labeled with α³²P dCTP using random primers essentially as previously described. The 2.4 kb c-myc message is indicated. Note that cells grown at 32.5° C. rapidly loss expression of c-myc mRNA.

This experiment eliminates one possible mechanism by which p53 induces apoptosis in cells. It has been previously reported that expression of c-myc in cell cycle arrested cells results in apoptosis. Thus, since p53 is known to block cells in Gi, it is possible that the p53-induced Gi cell block killed MEL cells because c-myc continued to be expressed. Instead, these data imply that p53 might exert at least some of its effects through downregulation of c-myc expression. This observation is consistent with our earlier finding that p53 accelerated death of MEL cells blocked in GI by agents other than p53. These data support a model in which p53 acts as a failsafe mechanism for cell cycle regulation.

### EXAMPLE XII

### C-myc blocks p53-induced Apoptosis

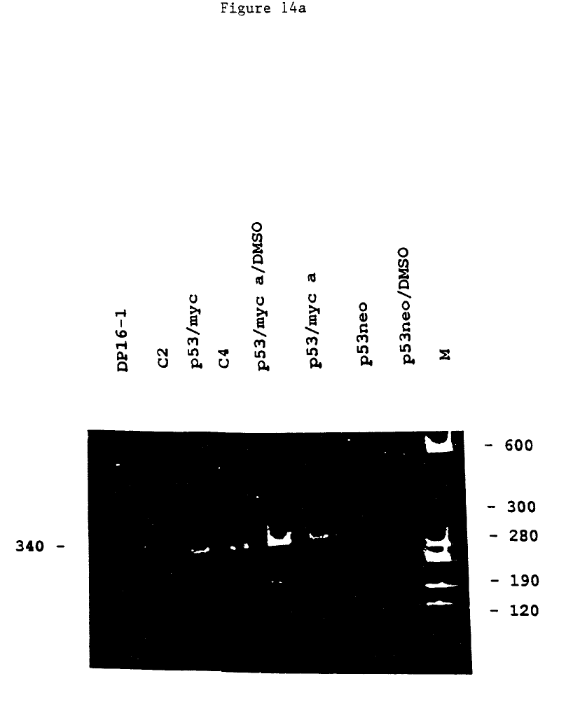

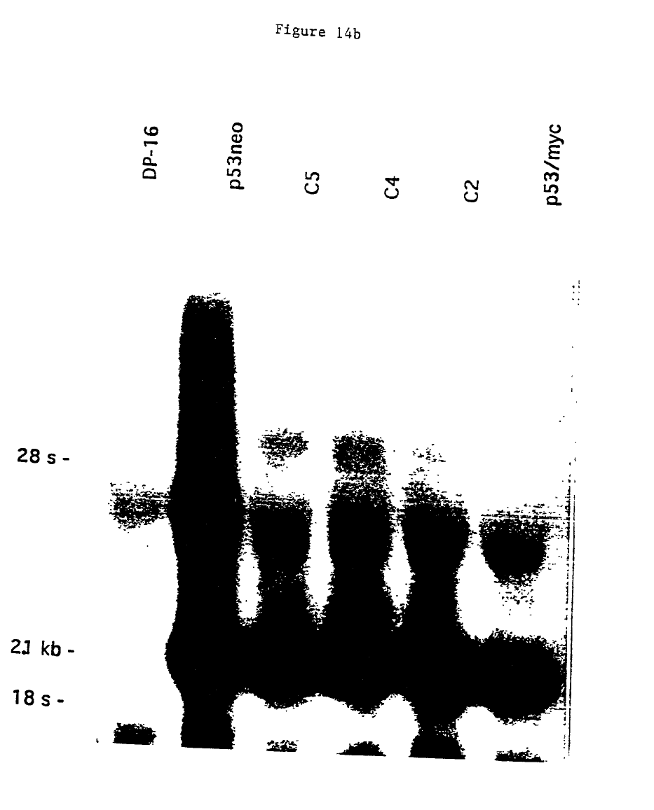

To determine whether c-myc modulates p53-induced effects, DP16-1 cells were transfected with p53^{ts}neo and pXVmyc/dhfr and selected cells by sequential growth in G418 and methotrexate. As a negative control, DP16-1 cells were cotransfected with p53^{ts}neo and pSVLZquad/dhfr (a vector in which the 4 leucines in the myc leucine zipper have been mutated). Total cellular RNA was isolated from the bulk culture and individual clones, and was analyzed by RT/PCR for expression of the transfected and mRNA (FIG. 14a) and by Northern blot for expression of p53 (FIG. 14b). The expression of p53 protein in these cells was confirmed by immunoprecipitation with antibodies directed to p53.

For FIG. 14a, one μg of RNA was transcribed into cDNA using MuLV RT (Superscript, BRL). One twentieth of the cDNA was amplified with TAQ DNA polymerase and a 3′ primer homologous to the c-myc cDNA and a 5′ primer homologous to transcribed SV40 sequences unique to the exogenous myc mRNA. The expected 310 bp fragment was present in the transfected clones, but not in the DP16-1 controls. Shown in FIG. 14c is twenty micrograms of RNA were analyzed by Northern blot The probe was the 3.5 kb p53 Sma fragment from p53^{ts}neo. As expected, all the transfected clones, but not the parental cell line, express p53.

### EXAMPLE XIII

### Conformation of p53^{ts} in DMSO-treated DP16-1 Cells

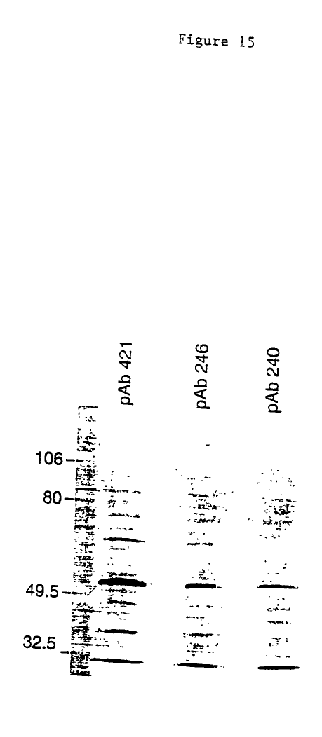

DMSO induces the ts mutant p53 to assume the wild-type phenotype, translocate to the nucleus, and causes cell death. Protein extracts from the DMSO-treated cells were probed with conformation specific antibodies, the majority of the p53^{ts} assumed the wild-type phenotype (FIG. 15). p53^{ts}neo co-transfected MEL cells were grown in medium containing 1.6% DMSO for 24 hours. Cells were then washed and resuspended in methionine-free medium containing 1.6% DMSO and ³⁵S methionine for three hours. Cells were harvested, and lysed in 10 mM Tris pH 7.5, 75 mM KCl, 1 mM EDTA, 0.25 mM PMSF, and 0.1% triton X-100. After removal of cellular debris, the protein extract was divided into equal aliquots and immunoprecipitated with p53 monoclonal antibody pAb421 (which recognizes both the wild-type and mutant conformation), pAb246 (which recognizes only the wild-type phenotype), and pAb240 (which recognizes only the mutant phenotype). Note that the majority of the newly synthesized p53 is in the wild-type conformation.

### EXAMPLE XIV

### Viability and Differentiation of DP16-1 Cells Transformed with p53 and c-myc

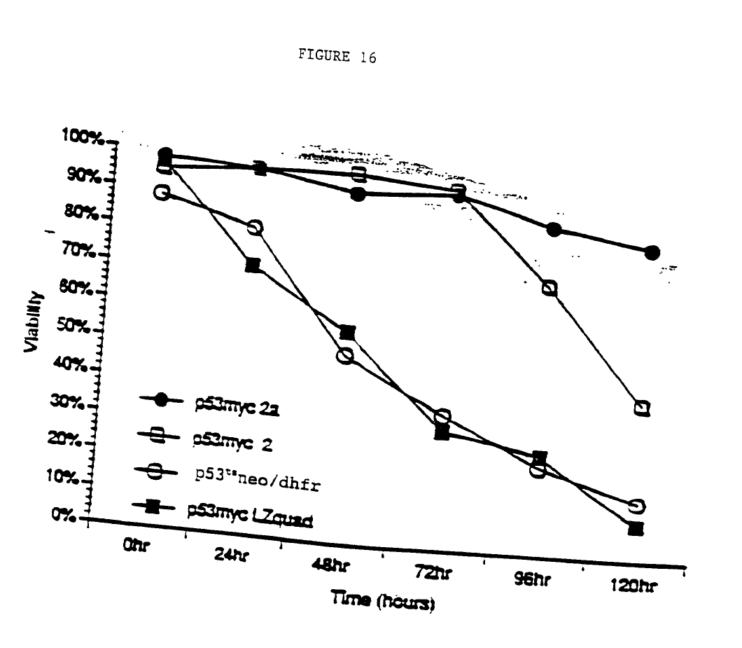

Amplified c-myc/p53^{ts}neo transfected bulk culture and several individual clones, as well as control pLZquad/dhfr/p53^{ts}neo transfected cells, were incubated in medium containing 1.6% DMSO. As expected, DP16-1 cells maintained viability and differentiated, and cells co-transfected with pLZquad/dhfr and p53 died. In contrast, cells transfected with both p53 and c-myc maintained viability (FIG. 16), but differentiated. Symbols presented in the figure are as follows: parental line DP16-1: p53^{ts}neo transfected DP16-1 cell bulk culture; [symbol] p53^{ts}neo and pSVmyc/dhfr cotransfected DP16-1 cell bulk culture; [symbol] p53^{ts}neo and pSVmyc/dhfr cotransfected DP16-1 cells; [symbol] p53^{ts}neo and pSVmyc/dhfr cotransfected DP16-1 cell bulk culture (amplified myc); and [symbol] p53^{ts}neo and pSVmycLZquad/dhfr (mutant myc) cotransfected DP16-1 cells.

Thus, cells transfected with myc, but not a mutant c-myc, behave in an analogous manner as cells transfected with c-myb and p53. At 0 hours DMSO was added to a final concentration of 1.6% to the medium of growing cells. Differentiation was monitored by benzidine staining. Note that the parental cell line maintained a high viability until cells became terminally differentiated, p53^{ts}neo of pSVmyc/dhfr cells rapidly died, and cells transfected with both plasmids has intermediate viability and differentiation patterns. DP16-1 cells cotransfected with pSV2neo or pSV2neo and pFR400 (dhfr) behaved in a manner identical to the parental cell line.

### EXAMPLE XV

### Enhancement of MEL Cell Differentiation by c-myc in the Absence of Mutant p53

MEL cell lines invariability contain mutations of the p53 tumor suppressor gene and express either negligible or mutant p53 protein. During MEL differentiation there is a biphasic decline in expression of the c-myc oncogene although enforced expression of c-myc blocks this differentiation. To determine the relationship between mutant p53 and c-myc in cell differentiation, DP16-1 cells (which have deleted both p53 alleles) were transfected with an amplifiable plasmid vector containing a full-length mouse c-myc cDNA with or without plasmid vector containing a mutant p53 cDNA (p53^{pro193}). The exogenous c-myc cDNA is transcribed in MEL cells and results in increased expression of c-myc mRNA and protein during DMSO-induced differentiation.

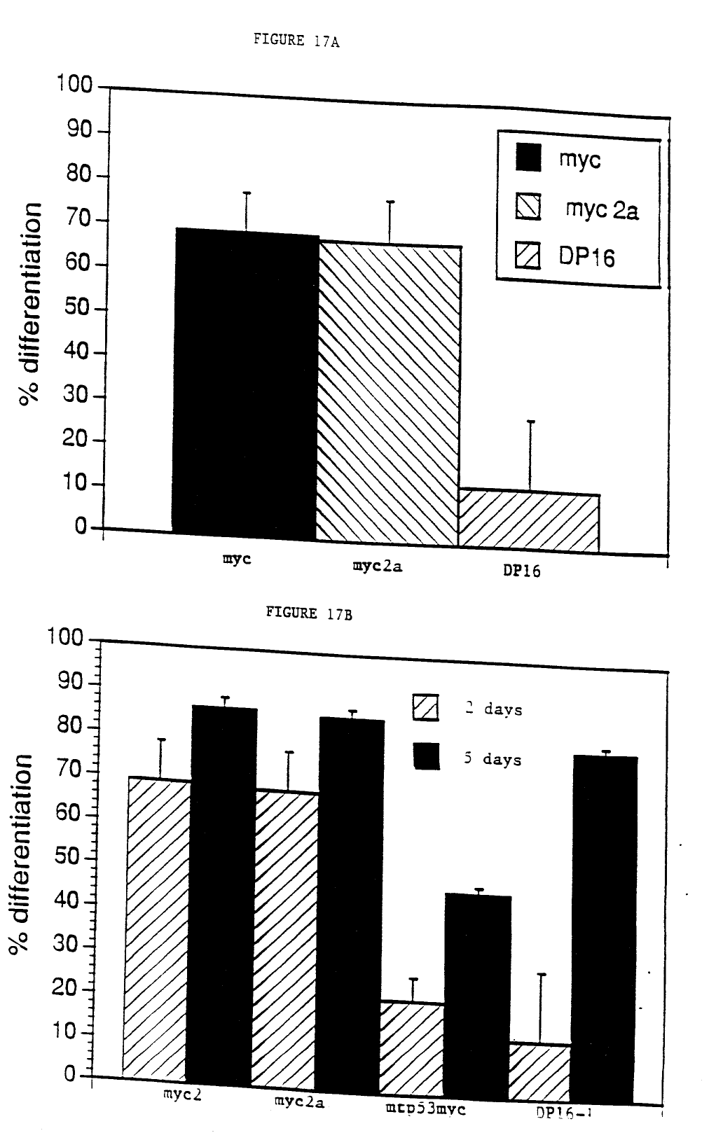

Extent of differentiation was monitored by benzidine staining (FIGS. 17A and 17B). When MEL cells are exposed to DMSO, differentiation normally occurs over a five day period. In contrast to the parental DP16-1 cells or DP16-1 cells transfected with pSV₂LZquad, c-myc transfected DP16-1 cells or underwent accelerated differentiation. The parental cells remained largely undifferentiated after two days of exposure to medium containing DMSO, while about 70-80% of the c-myc transfected cells were differentiated. Furthermore, c-myc transfected DP16-1 cells stopped growing and eventually lost viability when grown in medium containing 1.6% DMSO. Coincident expression of p53^{pro193} with c-myc abolished the accelerated DMSO-induced differentiation seen in cells which expressed c-myc alone.

These results show that rather than blocking differentiation, enforced expression of c-myc in the absence of p53 accelerates differentiation. The accelerated differentiation is reversed by coincident expression of mutant p53 with c-myc. Thus, enforced expression of c-myc can potentiate a differentiation signal and mutant p53 can overcome this potentiation.

### EXAMPLE XVI

### BCL2 Delays p53-induced Apoptosis

MEL cells were transfected with p53^{ts} hygro (constructed by replacing the p53^{ts}neo neomycin resistance gene with a hygromycin resistance gene) or p53^{ts} hygro and p^{asV}bc12neo. DP16-1 cells which express p53^{ts} or p53^{ts} and bc12 were selected. When wild-type p53 was analyzed by growing transfected cells at 32.5° C., the cells expressing p₅₃^{ts} rapidly die over a two day period. However, cells coexpressing p53^{ts} and bc12 maintain viability until day four.

### EXAMPLE XVII

### Purging CML Cells from Low Density Mononuclear Bone Marrow Cell With Antisense myb

CML cells often are selectively depleted during culture, this depletion, however, is variable and slow. Patients with CML can be treated by autologous bone marrow transplantation using hematopoietic cells which have been cultured in the presence of c-myb antisense oligodeoxynucleotides.

The most successful purging of bone marrow cells includes from one-to-three non-overlapping techniques. CML cells display antigens which are not present on the hematopoietic stem cells. A large majority of the CML cells that contaminant Marrow samples can initially be removed using antibodies that recognize these CML-specific antigens. Antibodies that can be used include specificities directed to one or more of the following antigens: CD15, CD33, Glycophorin A, CD10, CD2, CD4 and CD8. Antibody-cell conjugates can be removed by either flow cytometry, immuno-magnetic beads or compliment, for example. The remaining CML cells can be killed by incubating the enriched culture in the presence of c-myb antisense oligonucleotides.

Briefly, mononuclear cells will be isolated from a 5 ml bone marrow aspirate by centrifuging through a ficol-hypaque density gradient. The isolated bone marrow cells (1×10⁵ CD34+ progenitor cells) are then cultured in Dexter medium with IL3, SGF and EPO and macrophage inflammatory factor (MIP-1α; 300 μg/ml) for 24 hours and then are cultured in medium containing c-myb antisense oligonucleotides. MIP-1α arrests normal hematopoietic cells in G0 and these cells will therefore be resistant to c-myb antisense. The CML cells, however, are unresponsive to MIP-1α and will continue to cycle. For c-myb antisense treatment, the oligonucleotide 5′-GTGCCGGGGTCTTCGGGC-3′ is added at a concentration of 40 μg/ml and the culture is changed two times a day for a period of from 1-to-three days. The cells remaining viable in culture after this period will be only the normal hematopoietic cells.

The above treated cell culture is reinfused into the donor patient. This procedure effectively purges a bone marrow sample of cancer cells, while maintaining the viability of normal cells.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

We claim:

1. A method of reducing the viability of a proliferating mammalian cell deficient in p53 activity and in p53 suppressor activity of one or more p53-interacting regulatory proteins comprising increasing the level or activity of p53 in said cell.

2. The method of claim 1, wherein said proliferating mammalian cell is a cancer cell.

3. The method of claim 2, wherein said cancer cell is a leukemic cell, lymphoma cell, breast cancer cell, prostate cancer cell, oat cell carcinoma cell, lung cancer cell, colon cancer cell, bladder cancer cell, brain tumor cell or pancreatic cancer cell.

4. The method of claim 1, wherein said one or more p53-interacting regulatory proteins is c-myb, c-myc or bc12.

5. The method of claim 1, wherein said level or activity of p53 is increased by expressing a p53 encoding nucleic acid in said proliferating mammalian cell.

6. A method of reducing the viability of a proliferating mammalian cell exhibiting p53 activity and p53 suppressor activity of one or more p53-interacting regulatory proteins comprising reducing the suppressor activity of said one or more p53-interacting regulatory proteins.

7. The method of claim 6, wherein said proliferating mammalian cell is a cancer cell.

8. The method of claim 7, wherein said cancer cell is a leukemic cell, lymphoma cell, breast cancer cell, prostate cancer cell, oat cell carcinoma cell, lung cancer cell, colon cancer cell, bladder cancer cell, brain tumor cell, head and neck cancer cell or pancreatic cancer cell.

9. The method of claim 6, wherein said one or more p53-interacting regulatory proteins is c-myb, c-myc or bc12.

10. The method of claim 6, wherein said suppressor activity is reduced by decreasing the level of said one or more p53-interacting regulatory proteins.

11. The method of claim 10, wherein said level is decreased by introducing into said cell a nucleic acid substantially complementary in sequence to a nucleic acid encoding said one or more p53-interacting regulatory proteins.

12. The method of claim 11, wherein said nucleic acid substantially complementary in sequence to a p53-interacting regulatory protein encoding nucleic acid is an antisense oligonucleotide or analog.

13. A method of reducing the viability of a proliferating mammalian cell deficient in p53 activity and exhibiting p53 suppressor activity of one or more p53-interacting regulatory proteins, comprising:
(a) increasing the level or activity of p53 in said cell, and
(b) reducing the suppressor activity of said one or more p53-interacting regulatory proteins.

14. The method of claim 13, wherein said proliferating mammalian cell is a cancer cell.

15. The method of claim 14, wherein said cancer cell is a leukemic cell, lymphoma cell, breast cancer cell, prostate cancer cell, oat cell carcinoma cell, lung cancer cell, colon cancer cell, bladder cancer cell, brain tumor cell, head and neck cancer cell or pancreatic cancer cell.

16. The method of claim 13, wherein said level or activity of p53 is increased by expressing a p53 encoding nucleic acid in said proliferating mammalian cell.

17. The method of claim 13, wherein said one or more p53-interacting regulatory proteins is c-myb, c-myc or bc12.

18. The method of claim 13, wherein said suppressor activity is reduced by decreasing the level or activity of said one or more p53-interacting regulatory proteins.

19. The method of claim 18, wherein said suppressor activity is reduced by introducing into said cell a dominant-negative mutant of said one or more p53interacting regulatory proteins.

20. The method of claim 18, wherein said level is decreased by introducing into said cell a nucleic acid substantially complementary in sequence to a nucleic acid encoding said one or more p53-interacting regulatory proteins.

21. The method of claim 20, wherein said nucleic acid substantially complementary in sequence to a p53-interacting regulatory protein encoding nucleic acid is an antisense oligonucleotide or analog.

22. A method of selectively reducing the viability of proliferating cancer cells compared to nonproliferating normal cells within a mixed population of cells comprising selectively inducing p53-mediated apoptosis in said proliferating cancer cells.

23. The method of claim 22, wherein said cancer cell is a leukemic cell, lymphoma cell, breast cancer cell, prostate cancer cell, oat cell carcinoma cell, lung cancer cell, colon cancer cell, bladder cancer cell, brain tumor cell, head and neck cancer cell or pancreatic cancer cell.

24. The method of claim 22, wherein said p53-mediated apoptosis is selectively induced by increasing the level or activity of p53 in said proliferating cancer cells.

25. The method of claim 24, wherein said level or activity of p53 is increased by expressing a p53 encoding nucleic acid.

26. The method of claim 22, wherein said p53-mediated apoptosis is selectively induced by reducing the suppressor activity of one or more p53-interacting regulatory proteins.

27. The method of claim 26, wherein said suppressor activity is reduced by introducing into said cell a dominant-negative mutant of said one or more p53-interacting regulator proteins.

28. The method of claim 26, wherein said one or more p53-interacting regulatory proteins is c-myb, c-myc or bc12.

29. The method of claim 26, wherein said suppressor activity is reduced by decreasing the level of said one or more p53-interacting regulatory proteins.

30. The method of claim 29, wherein said level is decreased by introducing into said cell a nucleic acid substantially complementary in sequence to a nucleic acid encoding said one or more p53-interacting regulatory proteins.

31. The method of claim 30, wherein said nucleic acid substantially complementary in sequence to a p53-interacting regulatory protein encoding nucleic acid is an antisense oligonucleotide or analog.

32. The method of claim 22, wherein said p53-mediated apoptosis is selectively induced by increasing the level or activity of p53 in said proliferating cancer cells, and reducing the suppressor activity of said one or more p53-interacting regulatory proteins.

33. The method of claim 32, wherein said level or activity of p53 is increased by expressing a p53 encoding nucleic acid.

34. The method of claim 32, wherein said one or more p53-interacting regulatory proteins is c-myb, c-myc or bc12.

35. The method of claim 32, wherein said suppressor activity is reduced by decreasing the level or activity of said one or more p53-interacting regulatory proteins.

36. The method of claim 35, wherein said level is decreased by introducing into said cell a nucleic acid substantially complementary in sequence to a nucleic acid encoding said one or more p53-interacting regulatory proteins.

37. The method of claim 36, wherein said nucleic acid substantially complementary in sequence to a p⁵³-interacting regulatory protein encoding nucleic acid is an antisense oligonucleotide or analog.

38. A method of selectively reducing the viability of chronic granulocytic leukemia cells within a sample of proliferating bone narrow cells, comprising:
(a) selectively arresting the proliferation of normal cells within said sample of proliferating bone marrow cells to generate a sample of bone marrow cells containing arrested normal cells and proliferating chronic granulocytic leukemia cells, and
(b) inducing p53-mediated apoptosis in said proliferating chronic granulocytic leukemia cells.

39. The method of claim 38, wherein said proliferation of normal cells within said sample of proliferating bone marrow cells is selectively arrested by contacting said sample with an effective amount of a biologic response modifier.

40. The method of claim 39, wherein said biologic response modifier is MIP-1α.

41. The method of claim 38, wherein said p53-mediated apoptosis is induced in said proliferative chronic granulocytic leukemia cells by the method of claim 1, 6 or 13.

42. A method of inducing p53 tumor suppressor activity in a cell having a mutant p53 gene product, comprising treating the cell with an effective amount of DMSO sufficient to induce said mutant p53 gene product to assure a wild-type conformation or activity.

43. The method of claim 42, wherein said mutant p53 gene product is p53^{ts}.
